(19) United States
(12) Patent Application Publication
(10) Pub. No.: US 2009/0254056 A1
(43) Pub. Date: Oct. 8, 2009

(54) Device for collecting gravel and/or stones discharged from the urethra

(76) Inventor: Alejandro Fontes Munoz, Murcia (ES)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 10/584,684

(22) PCT Filed: Nov. 4, 2005
(86) PCT No.: PCT/ES05/00592

§ 371 Date: Jun. 10, 2009

(30) Foreign Application Priority Data
Nov. 4, 2004 (ES) U200402509

(51) Int. Cl. A61M 1/00

(52) U.S. Cl. 604/317

(57) ABSTRACT

The present invention relates to a device for collecting gravel and/or calculi expelled through the urethra, for example from a renal patient with kidney stones. The calculi and/or gravel can be collected comfortably and efficiently when they are expelled while urinating, wherever the patient may be, and to learn the approximate volume of gravel expelled. The patient can comfortably and discretely carry the device with him or her at anytime and in any place.

### DESCRIPTION OF THE INVENTION

The device proposed by the invention resolves in a fully satisfactory manner the drawbacks previously set forth in the different aspects discussed.

To that end and more specifically, said device is carried out as a type of sieve, based on a textile material with suitable porosity, finished at its lower end with a type of teat to facilitate collection, whereas at the level of its mouth, said sieve incorporates a preferably elliptical ring, carried out as a pair of rods which are in turn semi-elliptical, joined together in an articulated fashion at one of their ends, whereas at the other end they extend in straight sections for coupling to a complementary handle for holding the sieve. The articulated nature by means of which these two rods are joined together allows the folding of one over the other to minimize the volume of the device in an inoperative situation, in storage or in transport.

The straight section of one of these two rods is irremovably fixed to the handle, whereas the other one is fixed with rotational ability, having in its initial section close to the ring a flange that can be coupled in a groove of the handle or may remain outside of the latter, according to the position adopted by a moving proximal section of said handle, which is telescopically movable with respect to the rest of the handle by a magnitude that is in accordance with the length of said flange.

Locking means are arranged between the moving section of the handle and the fixed section thereof which keep said moving section perfectly stable in either of the two positions provided.

As a complement to the described structure and according to another feature of the invention, a housing is arranged in the fixed section of the handle, inside of which housing there is arranged a sample collection box, for which purpose said box has volumetric markings which allow determining the amount of sample collected, additionally having a lid for the hermetic sealing of said housing.

The device is complemented with an impermeable cover or case which allows internal housing of the duly folded device itself so that it can be carried by the user and used wherever needed.

### DESCRIPTION OF THE DRAWINGS

To complement the description being made and for the purpose of aiding to better understand the features of the invention, according to a preferred practical embodiment thereof, a set of drawings is attached as an integral part of said description, in which the following has been represented with an illustrative and non-limiting character:

FIG. 1 shows a perspective view of a device for collecting gravel and/or calculi expelled through the urethra, carried out according to the object of the present invention, which is shown in the use position and next to it an enlarged detail of the articulated joining between the two rods collaborating in the stiffening ring of the mouth of the sieve.

FIG. 2 also shows a perspective view of a partial detail of the device, at the level of its handle, with the latter in an operative situation and with its collecting deposit being open, in which an enlarged detail of the flange and grooves for locking said ring of the mouth of the funnel have also been shown.

FIG. 3 also shows a perspective view of the cover for housing the device of FIG. 1, which is part of the device as a whole, which is shown in an open situation.

FIG. 4 shows a plan view of the cover of the previous figure, also open, but with the collecting device itself duly housed inside it.

Finally, FIG. 5 shows a plan view of the assembly of the previous figure in a closed situation.

### PREFERRED EMBODIMENT OF THE INVENTION

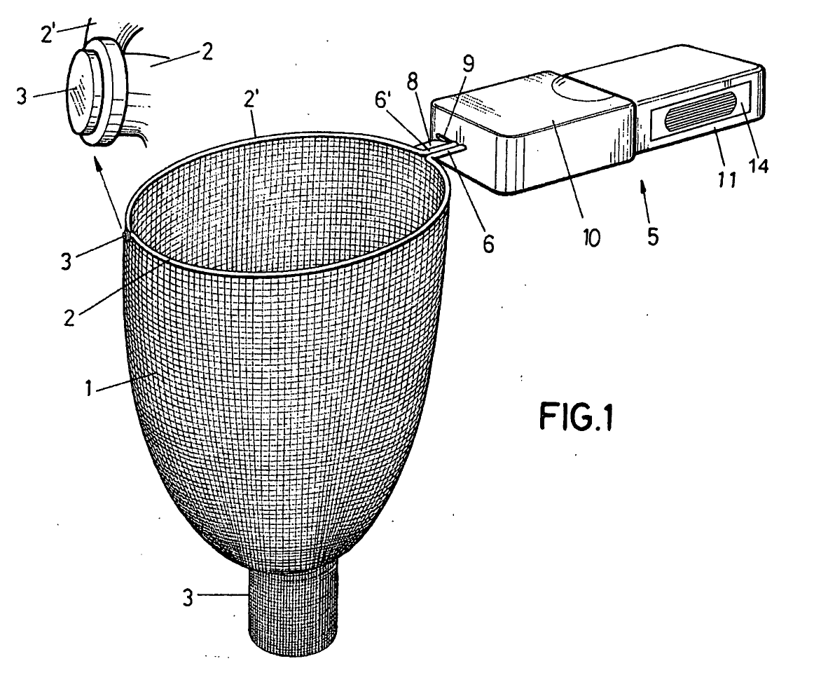

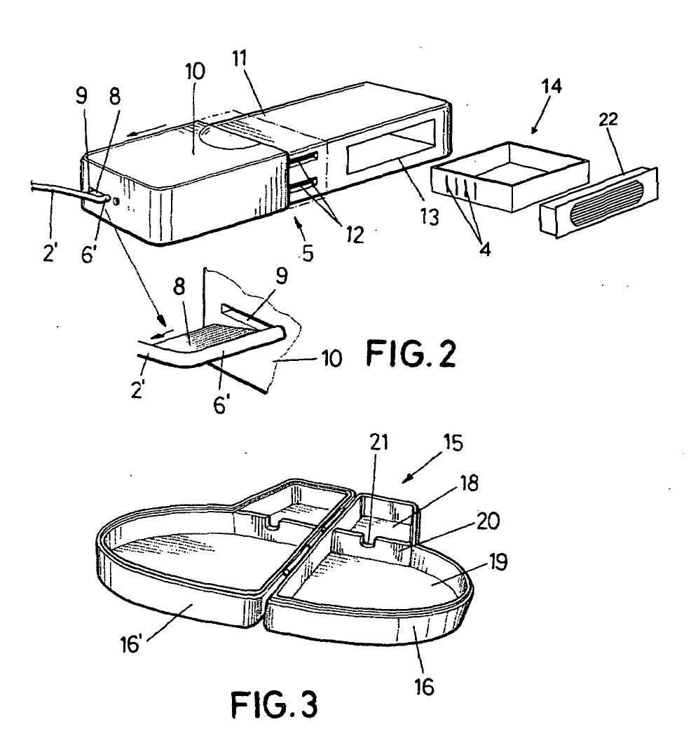

In view of the figures discussed it can be observed how the collecting device proposed by the invention is constituted of a sieve (1), of a textile nature and suitable porosity, particularly visible in FIG. 1 which, due to its own textile nature, is foldable and the mouth of which is stiffened by means of a pair of essentially identical rods (2-2′) which together form an ellipse with sufficient amplitude for allowing the passage therethrough of urine while the patient urinates.

The sieve (1) is finished at its lower end with a type of cylindrical teat (3) for quickly and easily collecting the amount of calculi and/or gravel expelled while urinating with a view to the adaptation thereof and subsequent supply of the corresponding data to the doctor controlling the renal patient.

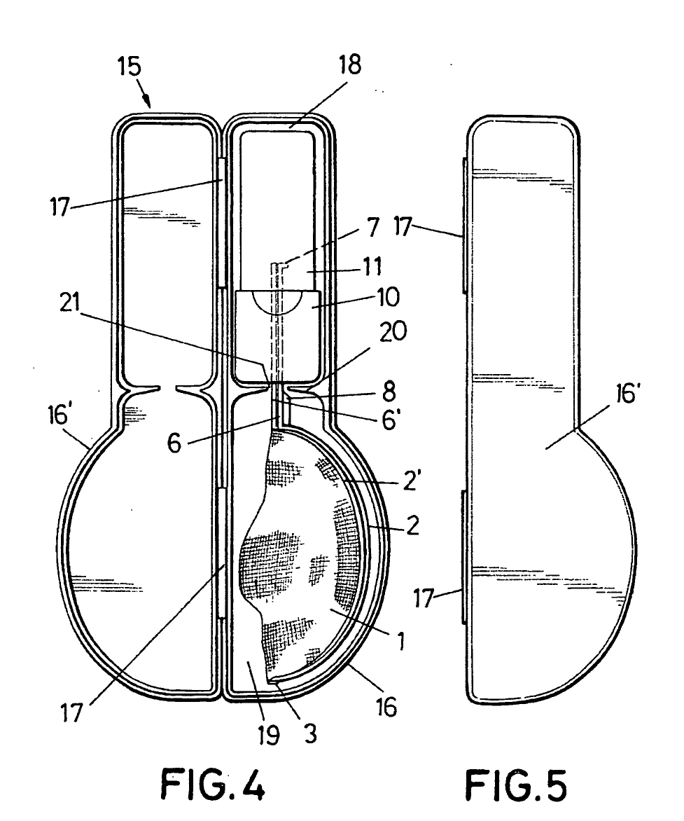

The rods (2-2′) stiffening the mouth of the sieve (1) are associated to a handle (5) to facilitate handling of the device and with a view to reducing its volume in an inoperative situation, as shown in FIG. 4, said rods (2-2′) are joined together in an articulated fashion at one of their ends (3), as is observed particularly in the enlarged detail of FIG. 1, whereas at their other end they extend in respective straight, parallel and very close sections (6-6′) entering the inside of a handle (5), one of which (6) is integrally joined to said handle, without movement ability, for example through a bend (7) on its free end, whereas the other one (6′) is assembled on said handle (5) with rotational ability in order to allow the folding of the ring (2-2′).

For said ring (2-2′) to be kept stable in an operative situation, as shown in FIG. 1, that is to keep the mouth of the sieve (1) open, it has been provided that the moving rod (2′), and more specifically the area in which its curved section (2′) and its straight section (6′) meet, incorporates a flange (8) complementary to a groove (9) existing in the handle (5), specifically in a proximal section (10), which is telescopically movable with respect to a fixed portion (11) of said handle, said fixed portion (11) having movement guides (12) for the moving section (10) provided with safety stops locking said proximal or moving section (10) in an operative situation of the device, ensuring that the ring (2-2′) stays open in such circumstances.

Finally, and as a complement of the described structure, a housing (13) is arranged in the fixed part (11) of the handle (5) for sample collection, inside of which there is arranged a sample collection box (14) which has a series of volumetric markings (4) for determining the amount of sample collected. Once the sample is collected, said box (14) is introduced in the housing (13) and said housing is subsequently hermetically sealed by means of a lid (22).

The collecting device itself, with the structure hereinbefore described, is housed in a cover or case (15) in which there is a pair of symmetrical shells (16-16′), hingedly joined together and with a single-piece nature with the cooperation of a hinge (17) and provided with any snap-fit closing means, one of said shells (16) being intended to receive the collecting device itself in a folded situation, as shown in FIG. 4, and the other one is intended to constitute a sealing lid, ensuring a perfect seal for said cover. Accordingly, in this sense there is arranged in each shell (16-16′) a section (18) which is suitably shaped and sized to receive the handle (5) and a section (19) in turn suitably shaped and sized to receive the ring (2-2′), with the sieve (1), duly folded, arranging an intermediate partition (20) between them which has a notch (21) on its upper edge, suitably arranged to allow passage of the rods (6-6′).

1. A device for collecting gravel and/or calculi expelled through the urethra during urination comprising a sieve of a textile nature and suitable porosity, said sieve having a mouth and provided at the level of said mouth an elliptical-shaped ring, said ring associated to a handle for handling the device, wherein said ring can be folded over itself to reduce the volume of the device in an inoperative situation.

2. The device according to claim 1, wherein said ring of the mouth of the sieve comprises two rods, each of said two rods comprising a curved section and a straight section, one of said two rods being joined to the other of said two rods in an articulated fashion at an end of a curved section, wherein each of said straight sections enter said handle, one of said curved sections being integrally joined to the other of the curved sections, to allow the swiveling of one of the curved sections with respect to the other of the curved sections.

3. The device according to claim 2, wherein said rod further comprises proximate said curved section said and straight section, a side flange for locking said rod side flange being lockable into a groove of said handle.

4. The device according to claim 1, wherein said sieve is finished at its lower end opposite said mouth with a cylindrical teat which allows collecting the expelled gravel and/or calculi.

5. (canceled)

6. (canceled)

7. The device according to claim 3, wherein said proximal section is arranged in said handle which is telescopically movable with respect to said handle, in which said groove is located, such that in a retracted position of said proximal section of the handle, said flange is located outside it, the swiveling of said rod being possible, whereas in the extended position of said section the flange is locked in said groove.

8. The device according to claim 4, further comprising a fixed section on said handle, said fixed section having a housing inside having a sample collection box, said sample collection box having a series of volumetric markings for determining the amount of sample collected, and a lid arranged for the hermetic sealing of said housing.

9. The device according to claim 3, further comprising a fixed section of the handle having movement guides for the moving section.

10. An assembly for collecting gravel and/or calculi expelled through the urethra, comprising device having a sieve of a textile nature and suitable porosity, said sieve having a mouth and provided at the level of said mouth an elliptical-shaped ring, said ring associated to a handle for handling the device, wherein said ring can be folded over itself to reduce the volume of the device in an inoperative situation and a case formed by two shells joined by means of a hinge, said case capable of receiving said device when said device is in a non-operable orientation.
